(19) United States
(12) Patent Application Publication
(10) Pub. No.: US 2011/0104774 A1
(43) Pub. Date: May 5, 2011

(54) RECYCLING AND REUSE OF CARBON DIOXIDE FOR TEMPERATURE CONTROL OF A FERMENTATION PROCESS

(76) Inventor: Carlos André de Oliveira Cardoso, Campinas-SP (BR)

(21) Appl. No.: 12/612,849

(22) Filed: Nov. 5, 2009

(51) Int. Cl. C12P 7/06; C12M 1/00

(52) U.S. Cl. 435/161; 435/303.1

(57) ABSTRACT

Methods and apparatus for improving the fermentation process by controlling the temperature of the fermentation tanks. Waste byproduct carbon dioxide from the fermentation process is recycled and used as a coolant medium for the fermentation process. These methods and apparatus are particularly useful for maintaining temperatures of the fermentation process when applied to production of green ethanol from sugarcane.

### FIELD OF THE INVENTION

The present invention relates to temperature control of fermentation processes, particularly those used for green energy production, using carbon dioxide (CO₂) produced during the fermentation. The present invention is particularly described with respect to the production of ethanol from the fermentation of sugarcane.

### BACKGROUND OF THE INVENTION

Fermentation is a common process used in a number of different industrial applications. One use of fermentation is to produce green energy ethanol from sugarcane. The general process for producing ethanol from sugar cane includes harvesting, milling, fermentation, distillation and dehydration. In particular, harvested sugarcane is transported to an ethanol production plant, where it is subjected to quality control, then washed, chopped and shredded. The resulting feedstock is then milled to collect a juice that contains ten to fifteen percent sucrose. This sugarcane juice is then filtered, chemically treated and pasteurized and then further filtered to produce a fluid rich in organic compounds. This fluid is evaporated resulting in a syrup that is then precipitated by a crystallization process to obtain clear sugar crystals and molasses. The sugar crystals are separated from the molasses by a centrifuge and the molasses is used for the production of ethanol.

The molasses is sterilized to remove impurities and is then ready for the fermentation process wherein the sugars are transformed into ethanol by the addition of yeast. The chemical equation for transformation of the molasses (sucrose) to ethanol is as follows:

C₁₂H₂₂O₁₁+H₂O→4C₂H₅OH+4CO₂

Fermentation time ranges from four to twelve hours providing an alcohol content of seven to ten percent by total volume (° GL), known as fermented wine. The best temperatures for the fermentation process are between 30° C. and 36° C. and working pressure is about atmospheric pressure. If the temperature drops below 12° C., no fermentation takes place. When the fermentation is complete, the yeast is recovered from the fermented wine using a centrifuge. The ethanol is then further treated to remove solids and to dehydrate to specified hydration standards required for fuel use. In particular, the alcohol is separated from the fermented wine by making use of the different boiling points between the alcohol and the main solid components. The resulting hydrated ethanol has a concentration of 96° GL and is the highest concentration of ethanol that can be achieved by azeotropic distillation. National specifications for ethanol allow up to 4.9% water by volume and therefore the hydrous ethanol is usually further dehydrated by chemical addition to achieve anhydrous ethanol of 99.7° GL. The ethanol produced from sugarcane provides energy that is renewable and less carbon intensive than oil and helps reduce air pollution and greenhouse gases because of cleaner emissions and because the growing sugarcane plants take up carbon dioxide from the air.

As noted above, there are significant green benefits achieved from the conversion of sugarcane to ethanol, there are also some drawbacks. In particular, the fermentation process is an exothermic chemical reaction which raises the temperature of the molasses, or syrup, being fermented. This increased temperature has an adverse affect on the yeast activity resulting in reduced ethanol production. In some cases, particular warmer climates, the increased temperature may kill the yeast thus stopping the fermentation process altogether. This leads to increased costs of materials because the yeast must be replaced and because of increased plant down time.

Therefore, there is a need in the art for improvements to fermentation processes in general and more particularly to the conversion of sugarcane to ethanol using fermentation.

### SUMMARY OF THE INVENTION

The present invention provides methods and apparatus for improving the fermentation process by controlling the temperature of the fermentation tanks. This is accomplished according to the present invention by recycling waste byproduct CO₂ from the fermentation process and using such as a coolant medium to control and maintain the temperature of the fermentation tanks and process.

### BRIEF DESCRIPTION OF THE DRAWINGS

FIG. 1 is a schematic drawing of an apparatus for controlling temperature of a fermentation process in accordance with one embodiment of the present invention.

### DETAILED DESCRIPTION OF THE INVENTION

The present invention defines methods and apparatus to improve fermentation processes through the control of the temperature of the fermentation tanks and process. The present invention particularly provides methods and apparatus that use recycled waste byproduct CO₂ from the fermentation process as a coolant medium to control and maintain the temperature of the fermentation tanks and process.

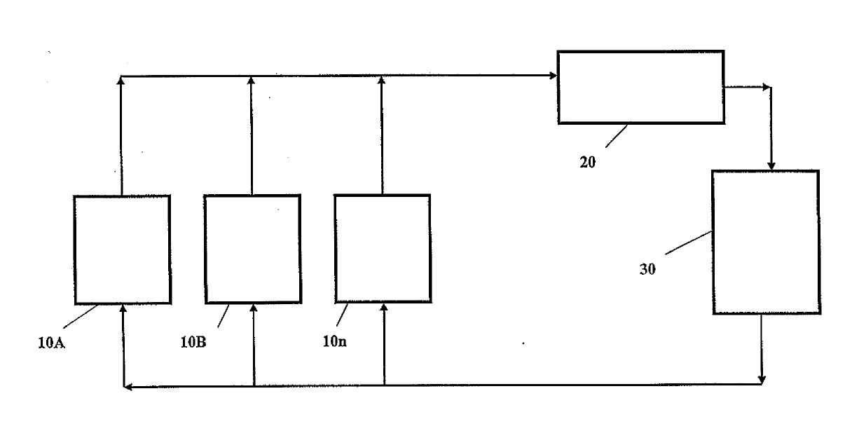

The present invention will be described more fully by referring to FIG. 1 that is a schematic drawing of an apparatus for controlling temperature of a fermentation process in accordance with one embodiment of the present invention. In particular, as shown in FIG. 1, a fermentation process having any number of fermentation tanks 10A, 10B, 10n, produces CO₂ gas as a byproduct during the fermentation process. This byproduct CO₂ gas is collected and then sent to a compressor 20 that compresses the CO₂ gas to liquid phase. The liquid CO₂ is then sent to a cryotank 30 for storage. The liquid CO₂ can then be returned to the fermentation process from the cryotank 30 as a cooling medium for the fermentation process.

By returning CO₂ to the fermentation process according to the present invention, temperature can be controlled within a relatively narrow range and more consistent and higher fermentation results can be achieved. This leads to better production results at lower costs. Further, by controlling the fermentation temperature, the risk of killing the yeast is nearly eliminated, thus keeping fermentation running at a higher rate and avoiding the extra cost of replacing yeast.

While the present invention may prove useful for any fermentation process, a particular embodiment of the present invention relates to the control of fermentation of sugarcane into ethanol. For such an application, the CO₂ created as the by-product of the fermentation process is used as a cooling medium to carefully control the temperature of fermentation. The CO₂ does not require any cleaning or processing (other than compression as noted above) prior to use as the cooling medium. The CO₂ can be injected directly into the fermentation tank or may be provided to a closed circuit, e.g. a pipe surrounding the fermentation tank. By providing the CO₂ in this manner to the fermentation process, it is possible to maintain the fermentation temperature in the optimum range of 30° C. to 36° C. to maximize the fermentation yield and to avoid harm to the yeast.

Benefits of the present invention are as noted above, e.g. better control of the fermentation process, higher fermentation yield, reduced or eliminated potential of harming or killing the yeast. Further, the present invention helps optimize and maximize the production of ethanol from sugarcane and thereby helps reduce dependence on fossil fuels and the emission of greenhouse gases from the combustion thereof.

It will be understood that the embodiments described herein are merely exemplary and that one skilled in the art may make variations and modifications without departing from the spirit and scope of the present invention. All such variations and modifications are intended to be included within the scope of the invention as described above. Further, all embodiments disclosed are not necessarily in the alternative, as various embodiments of the invention may be combined to provide the desired result.

What is claimed is:

1. A method of controlling the temperature of fermentation comprising:
collecting byproduct carbon dioxide gas created during fermentation;
compressing the carbon dioxide gas to obtain liquid carbon dioxide;
introducing the liquid carbon dioxide to the fermentation to maintain the temperature of fermentation.

2. The method of claim 1 wherein the fermentation is the fermentation of sugarcane to produce ethanol.

3. The method of claim 1 wherein the temperature of fermentation is maintained between 30° C. and 36° C.

4. The method of claim 1 wherein the fermentation runs for 4 to 12 hours.

5. The method of claim 1 wherein the liquid carbon dioxide is introduced directly to a fermentation tank in which the fermentation is occurring.

6. The method of claim 1 wherein the liquid carbon dioxide is introduced to a closed circuit surrounding a fermentation tank in which the fermentation is occurring.

7. An apparatus for fermentation that provides for temperature control comprising:
a fermentation tank in which fermentation will occur;
means to collect carbon dioxide gas created during the fermentation;
a compressor for compressing the carbon dioxide gas to obtain liquid carbon dioxide;
means for introducing the liquid carbon dioxide to the fermentation to control the temperature of fermentation.

8. The apparatus according to claim 7 wherein the fermentation is the fermentation of sugarcane to produce ethanol.

9. The apparatus of claim 7 wherein the temperature of fermentation is maintained between 30° C. and 36° C.

10. The apparatus of 7 wherein the means for introducing the liquid carbon dioxide to the fermentation is a means for injecting the liquid carbon dioxide directly to the fermentation tank.

11. The apparatus of claim 7 wherein the means for introducing the liquid carbon dioxide to the fermentation is a means for introducing the liquid carbon dioxide to a closed circuit surrounding the fermentation tank.

12. The apparatus of claim 8 further comprising a cryotank for storing the liquid carbon dioxide until the liquid carbon dioxide is provided to the fermentation.
